(19) United States
(12) Patent Application Publication
(10) Pub. No.: US 2003/0065000 A1
(43) Pub. Date: Apr. 3, 2003

(54) Method of treating cancer

(76) Inventors: Pamela Sarah Cohen, Tenafly, NJ (US); Howard Ashley Ball, Kendall Park, NJ (US); Christina Portrude Ravera, Chatham, NJ (US); Lucy Lee, Parsippany, NJ (US)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/235,251

(22) Filed: Sep. 5, 2002

Related U.S. Application Data
(60) Provisional application No. 60/361,656, filed on Mar. 5, 2002.
(60) Provisional application No. 60/332,400, filed on Nov. 16, 2001.
(60) Provisional application No. 60/317,594, filed on Sep. 7, 2001.

(51) Int. Cl. A61K 31/519

(52) U.S. Cl. 514/265.1

(57) ABSTRACT

This invention relates to an alternative regimen for the administration of a 7H-pyrrolo[2,3-d]pyrimidine derivative that are useful for the treatment of cancer. According to the inventive regimen the human patient receives a dose of the 7H-pyrrolo[2,3-d]pyrimidine on only about 40 to 71 percent of the days over the period that the treatment is carried out.

The present invention relates to a superior treatment regimen for the administration of a 7H-pyrrolo[2,3-d]pyrimidine derivative to a human subject for the treatment of cancer, particularly tumorous cancers, and other pathological conditions that are treatable with a epidermal growth factor receptor protein tyrosine kinase inhibitor. The inventive treatment regimen proposes to offer benefit by maintaining efficacy of the 7H-pyrrolo[2,3-d]pyrimidine derivative while reducing liver toxicity as measured by plasma liver enzyme levels.

7H-pyrrolo[2,3-d]pyrimidine derivatives useful for treating tumor diseases and other conditions are disclosed in U.S. Pat. No. 6,140,332, which is here incorporated by reference in its entirety. This U.S. patent discloses that the 7H-pyrrolo[2,3-d]pyrimidine derivatives are administered in the case of an individual having a body weight of about 70 kg at a daily dose from approximately 0.1 grams to approximately 5 grams, preferably from about 0.5 grams to 2 grams. However, this disclosure only suggests an appropriate daily dose and does not suggest that the H-pyrrolo{2,3-d}pyrimidine derivative should be administered on alternate days.

The present invention is based on the discovery that when a 7H-pyrrolo[2,3-d]pyrimidine derivative is administered to a human on a daily basis, the 7H-pyrrolo[2,3-d]pyrimidine derivative often accumulates in the body. It has been postulated that the accumulation of the 7H-pyrrolo[2,3-d]pyrimidine derivative in the body after repeated daily doses is responsible for liver enzyme elevation that is evident in some human subjects about three to four weeks after the initiation to the treatment.

According to the present invention, the accumulation of the 7H-pyrrolo[2,3-d]pyrimidine derivative in the subject, and thus the potential for liver enzyme elevation, is reduced by providing treatment on an alternative dosage regimen as disclosed herein.

According to the present invention, the 7H-pyrrolo[2,3-d]pyrimidine derivative is administered to the human subject less frequently than on a daily basis. In particular, this aspect of the present invention relates to a treatment regimen whereby the 7H-pyrrolo[2,3-d]pyrimidine derivative is administered on only about 40% to about 71% of the days.

As used herein, the expression “week” means seven consecutive days. Thus, a three week period is twenty-one consecutive days starting on any day of the calendar week. The day that the first dose is given is considered to be the first day of the week. Any discussion using calendar weeks is intended to be for illustrative purposes only.

In a first aspect, the present invention relates to a method of treating a human subject with a 7H-pyrrolo[2,3-d]pyrimidine derivative, which comprises administering the 7H-pyrrolo[2,3-d]pyrimidine derivative to the human subject from three to five times in each seven day period for a period of three weeks or longer.

In a specific regimen, the 7H-pyrrolo[2,3-d]pyrimidine derivative is administered at a frequency of from every other day over at least a three week period to three times a week for at least three weeks.

More specifically, this aspect of the present invention relates to a method of treating a human subject with a 7H-pyrrolo[2,3-d]pyrimidine derivative, which comprises administering the 7H-pyrrolo[2,3-d]pyrimidine derivative to the human subject three or four times a week on alternate days for a period of three weeks or longer.

In a specific embodiment, the 7H-pyrrolo[2,3-d]pyrimidine derivative is administered three times each week on alternate days, for example, on Monday, Wednesday and Friday of each week, for at least three weeks. Thus, the 7H-pyrrolo[2,3-d]pyrimidine derivative is administered every other day until three doses are given and the next dose is administered at the beginning of the following week.

Preferably, the dosage regimen is carried out through at least four or more weeks, for example 4, 5, 6, 7 or 8 weeks.

In an alternative embodiment of the present invention, the 7H-pyrrolo[2,3d]pyrimidine derivative is administered daily for a period of from one to three weeks, e.g. about two weeks, followed by a period of one to three weeks, e.g. about two weeks, wherein the agent is not administered. This cycle is repeated for from 1 to several cycles, for example, from 3 or 4 to 8 or more cycles.

The 7H-pyrrolo[2,3-d]pyrimidine derivative is advantageously administered to the human subject at a pharmaceutically effective dosage in the range of from about 50 mg to about 2000 mg on days when the 7H-pyrrolo[2,3-d]pyrimidine derivative is administered.

The compound (R)-4-(4-((1-phenylethyl)amino)-7H-pyrrolo[2,3-d]pyrimidin-6yl)-phenol, or a pharmaceutically acceptable salt therof, is the preferred 7H-pyrrolo[2,3-d]pyrimidine derivative.

Thus, the present invention relates a method for administering (R)-4-(4-((1phenylethyl)amino)-7H-pyrrolo[2,3-d]pyrimidin-6-yl)-phenol to a human subject, which comprises administering a pharmaceutically effective amount of the (R)-4-(4-((1-phenylethyl)amino)-7H-pyrrolo[2,3-d]pyrimidin-6-yl)-phenol, or a salt thereof, to the human subject over at least a three week period on only about 40% to about 71% of the days.

Thus, according to a first aspect, the present invention relates to a method for administering (R)-4-(4-((1-phenylethyl)amino)-7H-pyrrolo[2,3-d]pyrimidin-6-yl)-phenol to a human subject, which comprises administering a pharmaceutically effective amount of the (R)-4-(4-((1-phenylethyl)amino)-7H-pyrrolo[2, 3-d]pyrimidin-6-yl)-phenol, or a salt thereof, to the human subject three or four times a week on alternate days for a period of three weeks or longer.

Specifically, the present invention relates to a method for administering (R)-4-(4-((1-phenylethyl)amino)-7H-pyrrolo[2,3-d]pyrimidin-6-yl)-phenol to a human subject, which comprises which comprises administering a pharmaceutically effective amount of the (R)-4-(4-((1-phenylethyl)amino)-7H-pyrrolo[2,3-d]pyrimidin-6-yl)-phenol, or a salt thereof, to the human subject three times a week on alternate days for a period of three weeks or longer.

The inventive method further relates to the above described methods of administering (R)-4-(4-((1-phenylethyl)amino)-7H-pyrrolo[2,3-d]pyrimidin-6-yl)-phenol to a human subject wherein the weekly dosage regimen is carried out for a period of 3, 4, 5, 6, 7, or 8 weeks.

The inventive method especially relates to the above described methods of administering (R)-4-(4-((1-phenylethyl)amino)-7H-pyrrolo[2,3-d]pyrimidin-6-yl)-phenol to a human subject wherein the pharmaceutically effective dose of (R)-4-(4-((1-phenylethyl)amino)-7H-pyrrolo[2,3-d]pyrimidin-6-yl)-phenol, when administered, is in the range from about 50 mg to about 2000 mg, for example, about 450 mg to about 1500 mg doses or about 500 mg to about 1200 mg doses.

More particularly, in instances where (R)-4-(4-((1-phenylethyl)amino)-7H-pyrrolo[2,3-d]pyrimidin-6-yl)-phenol, or a pharmaceutically acceptable salt thereof, is administered three times each week on alternate days, for example, on Monday, Wednesday and Friday of each week, for at least three weeks, the drug is administered in a dose of about 450 mg to about 1500 mg, for example 600 mg, (on days when it is administered).

In the alternative embodiment, (R)-4-(4-((1-phenylethyl)amino)-7H-pyrrolo[2,3-d]pyrimidin-6-yl)-phenol is administered daily for from one to three weeks followed by for one to three weeks wherein (R)-4-(4-((1-phenylethyl)amino)-7H-pyrrolo[2,3-d]pyrimidin-6-yl)-phenol is not administered. This cycle is repeated for from 1 to several cycles, for example, from 3 or 4 to 8 or more cycles.

More specifically, the (R)-4-(4-((1-phenylethyl)amino)-7H-pyrrolo[2,3-d]pyrimidin-6-yl)-phenol is administered daily for about two weeks followed by about two weeks wherein (R)-4-(4-((1-phenylethyl)amino)-7H-pyrrolo[2,3-d]pyrimidin-6-yl)-phenol is not administered. This cycle is repeated for from 1 to several cycles, for example, from 3 or 4 to 8 or more cycles

If (R)-4-(4-((1-phenylethyl)amino)-7H-pyrrolo[2,3-d]pyrimidin-6-yl)-phenol is administered on a daily basis, an appropriate dose is in the range from 450 mg per day to 900 mg per day, such as 450 to 800 mg per day, for example 600 mg per day.

The inventive method especially relates to the above described methods of administering (R)-4-(4-((1-phenylethyl)amino)-7H-pyrrolo[2,3-d]pyrimidin-6-yl)-phenol to a human subject wherein the (R)-4-(4-((1-phenylethyl)amino)-7H-pyrrolo[2,3-d]pyrimidin-6-yl)-phenol is administered to treat a tumor condition, especially cancer.

The inventive dosage regimen applies to the use of 7H-pyrrolo[2,3-d]pyrimidine derivative, for example, (R)-4-(4-((1-phenylethyl)amino)-7H-pyrrolo[2,3-d]pyrimidin-6-yl)-phenol, alone, or as part of a combination treatment therapy wherein it is co-administered with one or more additional pharmaceutical products useful for treating tumors, especially cancerous tumors. For purposes of this application co-administered means that the patient is treated with both drugs according to the proper schedule for each, but not necessarily that both drugs are administered together at the same time. Thus, the H-pyrrolo{2,3-d}pyrimidine derivative, for example, (R)-4-(4-((1-phenylethyl)amino)-7H-pyrrolo[2, 3-d]pyrimidin-6-yl)-phenol, may be administered alone or in combination with other anticancer agents in accordance with the present inventive dosage regimen.

The 7H-pyrrolo[2,3-d]pyrimidine derivative, for example, (R)-4-(4-((1phenylethyl)amino)-7H-pyrrolo[2,3-d]pyrimidin-6-yl)-phenol, is administered to the subject by methods known in the art for administering pharmaceutical products, for example, orally, rectally or parenterally, preferably orally as a tablet or capsule formulation.

We claim:

1. A method for administering (R)-4-(4-((1-phenylethyl)amino)-7H-pyrrolo[2,3-d]pyrimidin-6-yl)-phenol to a human subject, which comprises administering a pharmaceutically effective amount of the (R)-4-(4-((1-phenylethyl)amino)-7H-pyrrolo[2,3-d]pyrimidin-6-yl)-phenol, or a salt thereof, to the human subject over at least a three week time period on only about 40% to about 71% of the days in the time period.

2. A method for administering (R)-4-(4-((1-phenylethyl)amino)-7H-pyrrolo[2,3-d]pyrimidin-6-yl)-phenol to a human subject, which comprises administering a pharmaceutically effective amount of the (R)-4-(4-((1-phenylethyl)amino)-7H-pyrrolo[2,3-d]pyrimidin-6-yl)-phenol, or a salt thereof, to the human subject three or four times a week on alternate days for a period of three weeks or longer.

3. A method of claim 2 wherein the weekly dosage regimen is carried out for a period of 3, 4, 5, 6, 7, or 8 weeks.

4. A method of claim 2 wherein the pharmaceutically effective dose is in the range from about 50 mg to about 2000 mg.

5. A method of claim 4 wherein the pharmaceutically effective dose is in the range from about 450 mg to about 1500 mg.

6. A method of claim 4 wherein the pharmaceutically effective dose is in the range from about 500 mg to about 1200 mg.

7. A method of claim 2 wherein the (R)-4-(4-((1-phenylethyl)amino)-7H-pyrrolo[2,3-d]pyrimidin-6-yl)-phenol is administered to treat a tumor condition.

8. A method of claim 7 wherein the (R)-4-(4-((1-phenylethyl)amino)-7H-pyrrolo[2,3-d]pyrimidin-6-yl)-phenol is administered as part of a combination treatment therapy with one or more additional pharmaceutical products useful for treating tumors.

9. A method for administering (R)-4-(4-((1-phenylethyl)amino)-7H-pyrrolo[2,3-d]pyrimidin-6-yl)-phenol to a human subject, which comprises which comprises administering a pharmaceutically effective amount of the (R)-4-(4-((1-phenylethyl)amino)-7H-pyrrolo[2,3-d]pyrimidin-6-yl)-phenol, or a salt thereof, to the human subject three times a week on alternate days for a period of three weeks or longer.

10. A method of claim 9 wherein the weekly dosage regimen is carried out for a period of 3, 4, 5, 6, 7, or 8 weeks.

11. A method of claim 9 wherein the pharmaceutically effective dose is in the range from about 50 mg to about 2000 mg.

12. A method of claim 11 wherein the pharmaceutically effective dose is in the range from about 450 mg to about 1500 mg.

13. A method of claim 12 wherein the pharmaceutically effective dose is in the range from about 500 mg to about 1200 mg.

14. A method of claim 9 wherein the (R)-4-(4-((1-phenylethyl)amino)-7H-pyrrolo[2,3-d]pyrimidin-6-yl)-phenol is administered to treat a tumor condition.

15. A method of claim 14 wherein the (R)-4-(4-((1-phenylethyl)amino)-7H-pyrrolo[2,3-d]pyrimidin-6-yl)-phenol is administered as part of a combination treatment therapy with one or more additional pharmaceutical products useful for treating tumors.

16. A method for administering (R)-4-(4-((1-phenylethyl)amino)-7H-pyrrolo[2,3-d]pyrimidin-6-yl)-phenol to a human subject, which comprises administering a pharmaceutically effective amount of the (R)-4-(4-((1-phenylethyl)amino)-7H-pyrrolo[2,3-d]pyrimidin-6-yl)-phenol, or a salt thereof, to the human subject daily for a period of from one to three weeks followed by a period of one to three weeks wherein the (R)-4-(4-((1-phenylethyl)amino)-7H-pyrrolo[2,3-d]pyrimidin-6-yl)-phenol, or a salt thereof, is not administered and this schedule is followed for one or more cycles.

17. A method of claim 16 wherein the (R)-4-(4-((1-phenylethyl)amino)-7H-pyrrolo[2,3-d]pyrimidin-6-yl)-phenol, or a salt thereof, is administered daily for a period of about two weeks followed by a period of about two weeks wherein the (R)-4-(4-((1-phenylethyl)amino)-7H-pyrrolo[2,3-d]pyrimidin-6-yl)-phenol, or a salt thereof, is not administered.

18. A method of claim 16 wherein the pharmaceutically effective amount of (R)-4-(4-((1-phenylethyl)amino)-7H-pyrrolo[2,3-d]pyrimidin-6-yl)-phenol is in the range from 450 mg to 800 mg per day.

19. A method of claim 16 wherein the (R)-4-(4-((1-phenylethyl)amino)-7H-pyrrolo[2,3-d]pyrimidin-6-yl)-phenol is administered to treat a tumor condition.

20. A method of claim 19 wherein the (R)-4-(4-((1-phenylethyl)amino)-7H-pyrrolo[2,3-d]pyrimidin-6-yl)-phenol is administered as part of a combination treatment therapy with one or more additional pharmaceutical products useful for treating tumors.
